(19) United States
(12) Patent Application Publication
(10) Pub. No.: US 2012/0213704 A1
(43) Pub. Date: Aug. 23, 2012

(54) SYSTEM AND METHOD FOR MOLECULAR IN VIVO IMAGING AND THERANOSTICS

(76) Inventors: Richard M. Awdeh, Miami Beach, FL (US); Victor L. Perez, Coconut Grove, FL (US); Adam De La Zerda, Woodside, CA (US); Sanjiv Gambhir, Portola Valley, CA (US)

(21) Appl. No.: 13/328,207

(22) Filed: Dec. 16, 2011

Related U.S. Application Data
(63) Continuation of application No. PCT/US2010/039159, filed on Jun. 18, 2010.
(60) Provisional application No. 61/218,284, filed on Jun. 18, 2009.

(51) Int. Cl. A61K 49/00; B82Y 15/00

(52) U.S. Cl. 424/9.1; 977/773; 977/902; 977/810; 977/762

(57) ABSTRACT

Systems and methods of characterizing biological tissues are provided. In the systems and methods, a first optical coherence tomography (OCT) image of a selected portion of biological tissues combined with a plurality of nanoparticles is obtained, where the plurality of nanoparticles are configured to bind to one or more types of biological molecules in the biological tissues and to produce contrast during OCT imaging. The systems and methods also include estimating a first distribution of the plurality of nanoparticles in the selected portion based on the first OCT image and characterizing the selected portion with respect to the types of biological molecules based on the distribution.

### CROSS REFERENCE TO RELATED APPLICATIONS

This application is a Continuation of, and claims priority under 35 U.S.C. §120 to, International Application No. PCT/US2010/039159, filed Jun. 18, 2010, which claims the benefit of U.S. Provisional Application No. 61/218,284, entitled “OPHTHALMIC MOLECULAR IN VIVO IMAGING AND THERANOSTICS UTILIZING METALLIC NANOPARTICLES”, filed Jun. 18, 2009, the entireties of which are incorporated by reference herein.

### FIELD OF THE INVENTION

This invention relates to systems and method for performing molecular in vivo imaging and theranostics.

### BACKGROUND

Molecular in vivo imaging allows a clinician or scientist the ability to perform real-time characterization and quantification of biological processes in vivo. This is accomplished by using a combination of an imaging device and a probe, which is localized to a molecular target, and can be detected by the imaging device. Forms of molecular in vivo imaging currently exist in SOME medical fields. These forms utilize the combination of existing imaging devices (including computed tomography scanning, magnetic resonance imaging, and positron emitting tomography (PET)) with specific probes or contrast agents which can be detected by these imaging modalities. For example, approaches to molecular in vivo imaging include bioluminescent imaging, in which light is generated by a chemiluminescent reaction triggered when an enzyme (e.g., luciferase from the North American firefly) binds to a substrate; magnetic resonance spectroscopy which uses the nuclei of elements (including carbon, fluorine, and phosphorus) to assess tissue energetic and cellular metabolism; magnetic nuclear isotopes which can be used to detect drug distribution and concentration; ultrasound imaging with contrast agents, including functionalized-microbubbles, which can be used to image tissue targets in the vascular component and therefore particularly useful in cardiovascular disease; and PET, which can be used to visualize tumor metabolism of various agents.

Such methods provide in vivo molecular imaging for various biological structures and tissues. However, in the case of some types of biological tissues, such as the eye, no effective methods for performing molecular in vivo imaging are generally available. Accordingly, such a limitation prevents treatment of many ophthalmic conditions until a phenotypic change can be seen.

### SUMMARY

Embodiments of the invention provide systems and method for characterizing biological tissues. In a first embodiment of the invention, a method of characterizing biological tissues is provided. The method includes obtaining a first optical coherence tomography (OCT) image of a selected portion of biological tissues combined with a plurality of nanoparticles, the plurality of nanoparticles configured to bind to one or more types of biological molecules in the biological tissues and to produce contrast during OCT imaging. The method also includes estimating a first distribution of the plurality of nanoparticles in the selected portion based on the first OCT image and characterizing the selected portion with respect to the types of biological molecules based on the distribution.

In a second embodiment of the invention, a method is provided for characterizing biological tissues combined with a plurality of nanoparticles configured to bind to one or more types of biological molecules in the biological tissues. The method includes obtaining a first intensity dataset for a first OCT image of a selected portion of the biological tissues using electromagnetic radiation with a center wavelength substantially equal to a peak absorption wavelength of a plurality of nanoparticles. The method also includes obtaining a second intensity dataset for a second OCT image of the selected portion using electromagnetic radiation with a center wavelength substantially unequal to a peak absorption wavelength of a plurality of nanoparticles. The method further includes generating a third intensity dataset defining a third OCT image of the selected portion based on a combination of the first and second intensity datasets, the third intensity dataset indicating the areas of the selected portion comprising one or more of the plurality of nanoparticles bound to the types of biological molecules.

In a third embodiment of the invention, a system is provided for characterizing biological tissues combined with a plurality of nanoparticles configured to bind to one or more types of biological molecules in the biological tissues and to produce contrast during OCT. The system includes a storage element for storing data associated with OCT images of a selected portion of the biological tissues and a processing element communicatively coupled to the storage element. In the system, the processing element is configured for estimating a first distribution of the plurality of nanoparticles in the selected portion based on the data associated with a first OCT image and characterizing the selected portion with respect to the types of biological molecules based on the first distribution.

In a fourth embodiment of the invention, a system is provided for characterizing biological tissues combined with a plurality of nanoparticles configured to bind to one or more types of biological molecules in the biological tissues and to produce contrast during optical coherence tomography (OCT). The system includes a storage element for storing a first intensity dataset for a first OCT image of a selected portion of the biological tissues obtained using electromagnetic radiation with a center wavelength substantially equal to a peak absorption wavelength of a plurality of nanoparticles and a second intensity dataset for a second OCT image of the selected portion obtained using electromagnetic radiation with a center wavelength substantially unequal to a peak absorption wavelength of a plurality of nanoparticles. The system also includes a processing element communicatively coupled to the storage element. In the system, the processing element configured for generating a third intensity dataset defining a third OCT image of the selected portion based on a combination of the first and second intensity datasets, the third intensity dataset indicating the areas of the selected portion comprising one or more of the plurality of nanoparticles bound to the types of biological molecules.

### BRIEF DESCRIPTION OF THE DRAWINGS

FIG. 1 is a schematic of an exemplary OCT system 100 that can be used in the various embodiments of the invention.

FIGS. 2A-2C show OCT in vivo images of mouse corneas under different imaging conditions using the OCT system of FIG. 1.

FIGS. 3A-3D are OCT in vivo images of mouse corneas for different dilutions of gold nanoparticles obtained using the OCT system of FIG. 1

FIG. 4 is a flowchart of steps in an exemplary method 400 for characterizing biological tissues in accordance with an embodiment of the invention.

FIG. 5 is a flowchart of steps in an exemplary method 500 for identifying OCT intensity data associated with nanoparticles in biological tissues.

FIG. 6 is a three-dimensional plot of OCT intensity as a function of position for first and second OCT images of a mouse cornea obtained as described above with respect to FIG. 5.

FIG. 7 is a schematic diagram of a computer system for executing a set of instructions that, when executed, can cause the computer system to perform one or more of the methodologies and procedures described herein.

### DETAILED DESCRIPTION

The invention is described with reference to the attached figures, wherein like reference numerals are used throughout the figures to designate similar or equivalent elements. The figures are not drawn to scale and they are provided merely to illustrate the instant invention. Several aspects of the invention are described below with reference to example applications for illustration. It should be understood that numerous specific details, relationships, and methods are set forth to provide a full understanding of the invention. One having ordinary skill in the relevant art, however, will readily recognize that the invention can be practiced without one or more of the specific details or with other methods. In other instances, well-known structures or operations are not shown in detail to avoid obscuring the invention. The invention is not limited by the illustrated ordering of acts or events, as some acts may occur in different orders and/or concurrently with other acts or events. Furthermore, not all illustrated acts or events are required to implement a methodology in accordance with the invention.

As described above, there are generally no effective methods of ophthalmic molecular in vivo imaging with clinical utility. Although there has been some preliminary work with bioluminescent imaging in animals for the visualization of retinoblastoma growth and metastasis, the low spatial resolution of bioluminescent imaging hinders it from being utilized for effective monitoring of diseases of the eye.

OCT is typically used as a method for obtaining high resolution imagery of structures of the eye and other biological tissues. OCT provides a quick, non-contact and non-invasive imaging modality based on the magnitude of backscattered light reflected from target tissues. This method has been intensively used in vivo and in vitro for quantitative and qualitative analysis of the posterior segment of the eye, including thickness measurements of the retina and nerve fiber layers in various conditions. OCT has also been used to image the anterior segment of the eye, including the thickness of the cornea and epithelium, corneal flap thickness after LASIK procedures, anterior chamber angle, and iris thickness. Further, using real-time OCT, tears on the ocular surface can be imaged dynamically. Additionally, OCT provides the advantage of producing non-contact images of the anterior segment of the eye in both static and dynamic conditions.

However, even though OCT provides a method of imaging and analyzing structures of the eye, there has been a general lack of interest and effort with respect to enhancing OCT techniques to provide molecular imaging. Specifically, there has been a general lack of interest and effort in developing contrast agents to enhance OCT in order to provide effective in vivo molecular imaging of biological tissues such as the eyes. As a result, OCT imaging is generally limited to in vivo, macro-scale inspection of the imaged tissues.

In view of the limitations of conventional OCT imaging methods, the various embodiments of the invention provide systems and methods for providing molecular in vivo imaging of the eye and other types of biological tissues using an enhanced OCT method. In particular, the various embodiments of the invention utilize OCT in combination with nanoparticles that provide contrast in OCT images in order to perform molecular in vivo imaging. These nanoparticles can be tagged with antibodies or other targeting component(s), thus allowing the nanoparticles to attach or bind to specific molecular targets within the ophthalmic or other types of biological tissues. Thus, an enhanced OCT image is produced, where the portions of the biological tissues associated with the molecular targets are identified. Additionally, the various embodiments of the invention also provide a method of processing such enhanced OCT images to estimate a distribution of the nanoparticles in the portion of the biological tissues and thereafter estimate a disease or treatment load.

Although the various embodiments of the invention will be primarily described with respect to imaging, diagnosis, and treatment of ophthalmic tissues, the invention is not limited in this regard. Rather, the various embodiments of the invention are equally applicable to the imaging, diagnosis, and treatment of any type of biological tissues. Such biological tissues can include any type of tissues within or excised from any type of organism. Further, such biological tissues can include both live and dead tissues.

The various embodiments of the invention therefore provide several advantages over existing OCT techniques. First, the ability to provide in vivo molecular imaging allows characterization, on a molecular level, of pathologic processes within the eye or other biological tissues. This in turn allows for molecular characterization of disease processes within the eye, thus allowing molecular diagnosis of disease and quantification or characterization of disease load or burden] Second, the various embodiments of the invention allow for throughput compound screening in disease processes, as molecular drug targets can be visually verified in vivo. Third, the various embodiments of the invention can also be configured to provide a means for delivering gene and drug therapies to the diseased biological tissues and therefore can be described as a theranostic. Further, the various embodiments of the invention provide a means for the visual detection of drug/gene levels in the biological tissues, following the respective administration of each. Therefore, this provides a quantifiable method to monitor drug/gene levels in the respective ophthalmic tissues, providing the treating clinician to more accurately provide treatment for the diseased tissues.

### Exemplary OCT System

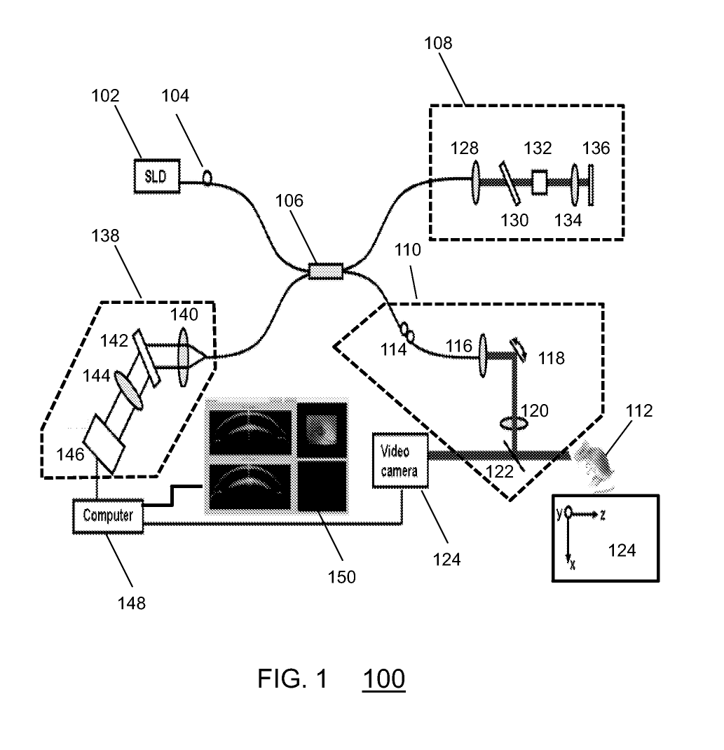

In the various embodiments of the invention, various types of OCT systems can be used to obtain OCT images. Types of OCT that can be used in the various embodiments of the invention, include, but are not limited to, time domain OCT and frequency domain OCT methods, such as spectral domain (SD) OCT. However, the various embodiments of the invention are not limited in this regard and any other OCT techniques no listed above can also be used. One exemplary system is illustrated in FIG. 1. FIG. 1 is a schematic of an exemplary OCT system 100 that can be used in the various embodiments of the invention. Although system 100 is shown in FIG. 1 as having a specific arrangement of components, the various embodiments of the invention are not limited in this regard. Rather, an OCT system in the various embodiments of the invention can have more or less components than those shown in FIG. 1.

As shown in FIG. 1, system 100 includes a light source 102 for producing low coherence light to be used to image a sample. As shown in FIG. 1, light source 102 can be a superluminescent diode (SLD) light source. However, the various embodiments of the invention are not limited in this regard and any other type of light source for producing low coherence light can be used in the various embodiments of the invention. As used herein, the term “low coherence light” refers to light with low temporal coherency (and high spatial coherency) so to allow high resolution imaging, such as the light can be emitted from superluminescent diodes. The light from light source 102 is directed though an optical isolator 104, such as a single mode optical fiber pigtail, to provide a low coherence light. However, the invention is not limited in this regard and other types of optical isolators can be used. The low coherence light, after passing through the optical isolator 104, is coupled to a fiber based Michelson interferometer. In particular, the low coherence light is directed through a fiber coupler/splitter 106, which directs a portion of the low coherence light (reference light) into the reference arm 108 and another portion (sample light) into the sample arm 110.

The sample arm 110 provides an X-Y galvanometer scanner and the optics for transporting the sample light to a sample 112 (i.e., the biological tissue). In the configuration shown in FIG. 1, the sample arm 110 consists of a polarization controller 114, a first sample lens 116, a X-Y scanning mirror 118, a second sample lens 120, and a beamsplitter 122 for directing the sample light to the sample 112. Additionally, as shown in FIG. 1, a motion stage 124 can be provided for positioning and aligning the sample 112 (i.e., the biological tissues of interest) with a path of the sample beam. Further, the sample 112 can be aligned and/monitored using a portion of the light reflected from the sample 112 and directed by beamsplitter 122 into a camera 126 or other imaging device. The other portion of the light is reflected back through components 114-120 to fiber coupler 106.

The reference arm 108, as shown in FIG. 1, includes a first reference lens 128, a neutral density filter 130, a dispersion compensator 132, a second reference lens 134, and a stationary mirror 136. Accordingly, the reference light is directed through the components of reference arm 108 and reflected back to fiber coupler 106. The reflected reference light and sample light are then combined at fiber coupler 106. The combined light is then directed into detection arm 138.

The detection arm 138, as shown in FIG. 1, includes a first detector lens 140 and a diffraction grating 142. In one embodiment, the diffraction grating 142 can be a high volume diffraction grating. However the various embodiments of the invention are not limited in this regard and other types of diffraction gratings can be used. Detection arm 138 also includes a second detector lens 144 and a sensor 146, as shown in FIG. 1. In one embodiment the sensor 146 can be a charge-coupled device (CCD), however, the various embodiments of the invention are not limited in this regard. For example, the sensor 146 can be any type of photodetector device(s), photomultiplier(s), or any other device capable to generating an electrical signal in response to incident light.

The image data collected at sensor 146 is then transmitted to computer 148. The computer 148 can then generate OCT images and cause a display 150 to present the OCT images to a user. In the configuration illustrated in FIG. 1, data from the imaging device 126 can also be processed by computer 148 and presented separately or concurrently with the OCT images. Further, computer 148 can also be configured to perform one or more of the processes or methods described below.

### Nanoparticle Configuration and Use

As described above, a first aspect of the invention is the combination of OCT imaging with nanoparticles. That is, the particles on having dimension less than 1000 nm are combined with the biological tissues of interest to provide a contrast agent for the OCT image. In the various embodiments of the invention, the nanoparticles can be combined with the biological tissues of interest in several ways. For example, the nanoparticles can be applied topically to an area of interest. In another example, the nanoparticles can be injected into the bloodstream (intravenous) or directly into the biological tissues of interest. Also, in the case of ophthalmic applications, the nanoparticles can be directly into the eye's vitreous space via an intravitreal injection.

As described above, the nanoparticles, first and foremost, act as a contrast agent during OCT imaging. Accordingly, in the various embodiments of the invention, a variety of configurations for the nanoparticles can be used that result in a signal being generated during OCT imaging. Specifically, different combinations of sizes, shapes, and compositions can be used in the various embodiments of the invention.

With respect to size and shape, the nanoparticles can be configured to comprise nanorods (i.e., nanoparticles that are generally cylindrical). In some embodiments of the invention, the nanorods can be configured to have an average length between 20 nm and 60 nm, such as 40 nm, and am average diameter or width between 5 nm and 20 nm, such as 10 nm. Accordingly, the aspect ratio of such nanorods can vary from 2 to 6. However, the aspect ratio and dimensions of the nanorods can vary according to fabrication techniques for the nanorods. Some conventional nanorod fabrication techniques can result in a variation of the aspect ratio of such nanorods from 1 to 1000, although a range of 2 to 6 will generally be more typical. However, the various embodiments of the invention are not limited solely to a nanorod shape for the nanoparticles. Rather, various other shapes with similar dimensions can be used in the various embodiments of the invention.

With respect to composition, the nanoparticles can consist of metals, semiconductors, small molecules, or any combination thereof. Metals for the nanoparticles can include gold, silver, iron-oxide, nickel, cobalt, and alloys thereof, to name a few. However, the various embodiments of the invention are not limited in this regard and other metals or alloys thereof can also be used. Semiconductors can include column IV semiconductors (e.g., silicon, germanium, or carbon) or compound semiconductors (e.g., gallium nitride, gallium arsenide, silicon carbide, silicon germanium), to name a few. In some embodiments, these semiconductor-based nanoparticles can formed using quantum dots. In addition, the nanoparticles can consist of small organic molecules (typically <1 nm in size).

In the various embodiments of the invention, the size, shape, and composition of the nanoparticles is selected to provide high optical absorption during the OCT process to provide the necessary contrast in the OCT image. For example, in the case of OCT performed using a light source with a center wavelength between ultraviolet and near-infrared wavelengths, the nanoparticles can be selected to have an optical extinction coefficient between 1×10⁵ M⁻¹cm⁻¹ and 1×10¹¹ M⁻¹cm⁻¹ for wavelengths between 500 and 1500 nm.

Additionally, the nanoparticles can be treated in the various embodiments of the invention to improve their water-solubility, thereby preventing their aggregation upon administration to a tissue or the blood stream. For example, the nanoparticles can be coated with, for example, polyethylene-glycol-5000 (PEG₅₀₀₀), to render them water soluble. However, the various embodiments of the invention are not limited in this regard and the nanoparticles can be coated with other substances to render then water soluble.

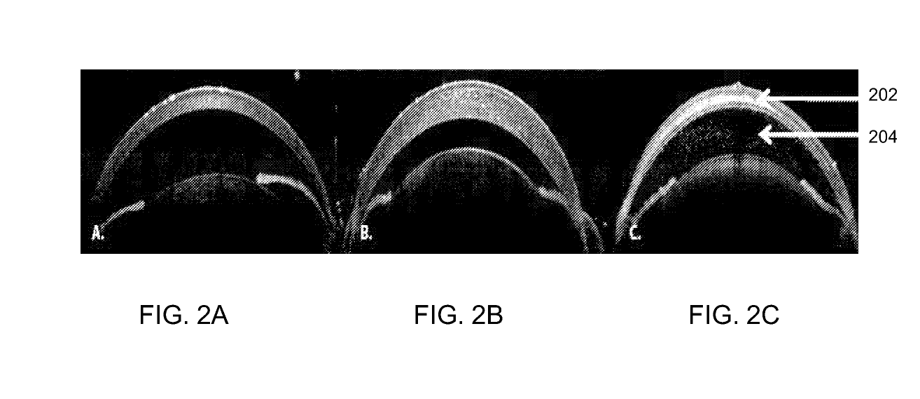

In one particular embodiment, the nanoparticles are gold nanoparticles shaped as nanorods, with an average diameter of 10 nm and an average length of 40 nm. As a result, the average aspect ratio of these gold nanorods is ˜4. However, this range can typically range from 2-6, as described above. Consequently, the optical extinction coefficient of such gold nanorods is ˜6.7×10⁸ M⁻¹cm⁻¹ and results in high optical absorption at wavelengths between 750 nm and 800 nm, such as 780 nm. Such nanoparticles are clearly detectable using the OCT configuration described above with respect to FIG. 1. This is shown below in FIGS. 2A-2C.

FIGS. 2A-2C show OCT in vivo images of mouse corneas under different imaging conditions using an OCT system with a light source having a center wavelength of 840 nm and a bandwidth of 100 nm. FIG. 2A shows an OCT image of naive mouse cornea. FIG. 2B shows and OCT image of a mouse cornea with a corneal intrastromal injection of 0.1 cc of a balanced salt solution (BSS) with no nanoparticles. As shown in FIG. 2B, the injection of BSS results in increased corneal edema, but no localized increase in intensity in the OCT image as compared to FIG. 2A. FIG. 2C shows an OCT image of a mouse cornea following intrastromal injection of 0.1 cc of 50 nM gold nanorods. The gold nanorods are configured as described above. As a result of the strong absorption of the gold nanorods, areas of increased signal intensity are observed in FIG. 2C, as compared to FIGS. 2A and 2B. In particular, a first area of increased signal intensity correlating to site of injected GNPs in the cornea stroma (indicated by arrow 202) and in the anterior chamber (indicated by arrow 204).

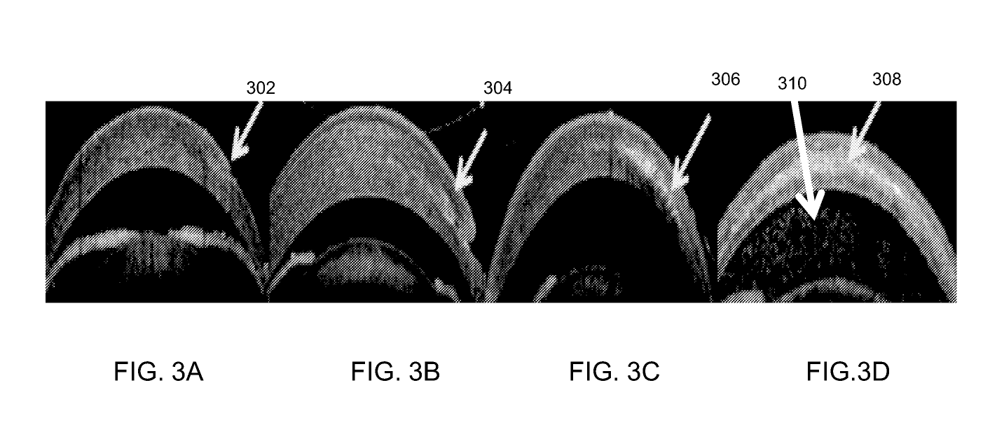

Further, as the concentration of nanoparticles is increased, the signal intensity is increased in the OCT image. This is illustrated in FIGS. 3A-3D. FIGS. 3A-3D are OCT in vivo images of mouse corneas for different dilutions of gold nanoparticles (GNPs). In particular, each of the images was generated by imaging a mouse eye having a 0.1 cc intrastromal injection, with 1:1000, 1:100, 1:10, and 1:1 dilutions of 50 nM concentration gold nanorods for FIGS. 3A, 3B, 3C, and 3D, respectively. In FIGS. 3A-3D, areas of increased signal intensity, correlating to site of injected GNPs, are marked with arrows 302-308, respectively. Note that FIG. 3D also shows GNPs in the mouse corneal stroma (arrow 308) as well as in the anterior chamber of the eye (arrow 310). Accordingly, FIGS. 3A-3D show that intensity varies with nanoparticle concentration. Specifically, FIGS. 3A-3D show that intensity varies generally linearly, thus allowing intensity data in OCT image to be translated into the nanoparticle concentration data. For example, in the cornea, a concentration of 1.4 nM of gold nanorods has produced OCT contrast of 6, or a signal to background ratio (SBR) of 6 for every 1.4 nM of gold nanorods concentration.

As described above, the nanoparticles are conjugated with a targeting molecule in order to allow the nanoparticles to bind to a particular biological molecule. In some embodiments, the nanoparticles can be multivalent. That is, more than one copy of type of targeting molecule may be conjugated with a nanoparticle. For example, a targeting molecule can be selected that allows the nanoparticle to bind to biological molecules such as proteins, receptors, enzymes, cells, genes, bacteria, or a viruses, to name a few. These can include, for example, anti-angiogenic factors, neural factors, tumor antigens and anti-inflammatory factors. Specific examples include In an embodiment wherein a particular type of cell (e.g., a cancer cell) is being targeted, a nanoparticle as described herein can be conjugated to a targeting molecule that specifically targets the particular type of cell. For example, if the cell being targeted overexpresses a particular receptor, or expresses a receptor not found on other cell types, a nanoparticle as described herein can be conjugated to a ligand that specifically binds the receptor. In another example, a nanoparticle as described herein can be conjugated to an antibody or antibody fragment that specifically binds an epitope expressed on the cell to be targeted. Antibody fragments that recognize and bind to specific epitopes on a cell are thus useful as a cell-specific targeting molecule can be generated by known techniques. For example, such fragments include but are not limited to F(ab′)₂ fragments that can be produced by pepsin digestion of the antibody molecule, and Fab fragments that can be generated by reducing the disulfide bridges of F(ab′)₂fragments. Human antibodies against a particular antigen or cell-surface receptor can be made by adapting known techniques for producing human antibodies in animals such as mice. See, e.g., Fishwild, D. M. et al., Nature Biotechnology 14 (1996): 845-851; Heijnen, I. et al., Journal of Clinical Investigation 97 (1996): 331-338; Lonberg, N. et al., Nature 368 (1994): 856-859; Morrison, S. L., Nature 368 (1994): 812-813; Neuberger, M., Nature Biotechnology 14 (1996): 826; and U.S. Pat. Nos. 5,545,806; 5,569,825; 5,877,397; 5,939,598; 6,075,181; 6,091,001; 6,114,598; and 6,130,314. Humanoid or humanized antibodies against a particular antigen can be made from non-human antibodies by adapting known methods such as those described in U.S. Pat. Nos. 5,530,101, 5,585,089, 5,693,761, and 5,693,762.

A targeting molecule as described herein can be naturally occurring, synthetically produced, or a combination thereof. Any suitable targeting molecule can be used. Examples of targeting molecules include antibodies, antibody fragments, epitopes, ligands, minibodies, diabodies, aptamers, affibodies, peptides, proteins, siRNA, peptide/nucleic acid conjugates, etc. However, the various embodiments of the invention are not limited in this regard and other targeting molecules can be used in the various embodiments to bind the nanoparticle to a wide range of biological molecules (targeting the biological molecules), including the biological molecules listed above.

The targeting component can be conjugated with the nanoparticles. The method for this conjugation greatly depends on the specific two molecules, but is typically a simple carboxyl-amine conjugation, such as described in greater in detail in Liu et al, Nat Nano 2007. As used herein, the term “conjugated” refers to when one molecule or agent is physically or chemically coupled or adhered to another molecule or agent. Examples of conjugation include covalent linkage and electrostatic complexation. The terms “complexed,” “complexed with,” and “conjugated” are used interchangeably herein. Nanoparticles are typically conjugated to at least one (e.g., 1, 2, 3, 4, 5, etc.) targeting molecule for targeting a particular biological molecule (e.g., cell). In these embodiments, nanoparticles can be prepared and conjugated to a cell-specific targeting molecule, for example, using any suitable method. Generally, a nanoparticle is conjugated to or bound to a targeting molecule by a chemical reaction such as carboxyl-amine reaction, typically. In some embodiments, conjugation or attachment of a targeting molecule is done with suitable spacers to preserve the binding properties of the targeting molecule (e.g., peptide, protein, antibody). A linker or spacer molecule may be used in conjugating a targeting molecule to the nanoparticles described herein. As used herein, the terms “linker” or “spacer” mean the chemical groups that are interposed between the nanoparticle and the surface-exposed molecule(s) (i.e., the targeting molecule). The terms “displayed” or “surface exposed” are considered to be synonyms, and refer to targeting molecules (e.g., epitopes, antigen-binding fragments, antibodies, etc.) or other molecules that are present at the external surface of a structure such as a nanoparticle. Preferably, linkers are conjugated to the surface molecule at one end and at their other end to the nanoparticle. Linking may be performed with either homo- or heterobifunctional agents, i.e., SPDP, DSS, SIAB. Methods for linking are disclosed in PCT/DK00/00531 (WO 01/22995) to deJongh, et al., which is hereby incorporated by reference in its entirety.

The nanoparticles and compositions including the nanoparticles described herein may be administered to mammals (e.g., dog, cat, pig, horse, rodent, non-human primate, human) in any suitable formulation. For example, a composition including nanoparticles and a therapeutic agent (e.g., a drug, cell-specific targeting molecule, cytotoxin, etc.) may be formulated in pharmaceutically acceptable carriers or diluents such as physiological saline or a buffered salt solution. Suitable carriers and diluents can be selected on the basis of mode and route of administration and standard pharmaceutical practice. A description of exemplary pharmaceutically acceptable carriers and diluents, as well as pharmaceutical formulations, can be found in Remington's Pharmaceutical Sciences, a standard text in this field, and in USP/NF. Other substances may be added to the compositions to stabilize and/or preserve the compositions. Nanoparticles and compositions including nanoparticles as described herein can be used prophylactically as well as to treat a subject currently suffering from a disease or condition.

The nanoparticles and compositions including nanoparticles as described herein may be administered to mammals by any conventional technique. Typically, such administration will be parenteral (e.g., intravenous, subcutaneous, intratumoral, intramuscular, intraperitoneal, or intrathecal introduction). The compositions may also be administered directly to a target site (e.g., ophthalmic tissue such as cornea, vitreous, retina, subretinal space, choroid, etc.). The compositions may be administered in a single bolus, multiple injections, or by continuous infusion (e.g., intravenously, by peritoneal dialysis, pump infusion). For parenteral administration, the compositions are preferably formulated in a sterilized pyrogen-free form.

The nanoparticles and compositions including nanoparticles as described herein are preferably administered to a mammal (e.g., dog, cat, pig, horse, rodent, non-human primate, human) in an effective amount, that is, an amount capable of producing a desirable result in a treated mammal (e.g., molecular imaging of ophthalmic tissue, treatment of an ophthalmic disease or condition, etc.). Such a therapeutically effective amount can be determined as follows. Toxicity and therapeutic efficacy of the nanoparticles and compositions including nanoparticles as described herein can be determined by standard pharmaceutical procedures, using either cells in culture or experimental animals to determine the LD₅₀ (the dose lethal to 50% of the population). The dose ratio between toxic and therapeutic effects is the therapeutic index and it can be expressed as the ratio LD₅₀/ED₅₀. Those compositions that exhibit large therapeutic indices are preferred. The dosage of preferred compositions lies preferably within a range that includes an ED₅₀ with little or no toxicity. The dosage may vary within this range depending upon the dosage form employed and the route of administration utilized.

As is well known in the medical and veterinary arts, dosage for any one subject depends on many factors, including the subject's size, body surface area, age, the particular composition to be administered, time and route of administration, general health, and other drugs being administered concurrently.

By the phrases “therapeutically effective amount” and “effective dosage” is meant an amount sufficient to produce a therapeutically (e.g., clinically) desirable result; the exact nature of the result will vary depending on the nature of the disorder being treated. For example, where the disorder to be treated is cancer, the result can be elimination of cancerous cells including cancerous tumors. The compositions and vaccines described herein can be administered from one or more times per day to one or more times per week. The skilled artisan will appreciate that certain factors can influence the dosage and timing required to effectively treat a subject, including but not limited to the severity of the disease or disorder, previous treatments, the general health and/or age of the subject, and other diseases present. Moreover, treatment of a subject with a therapeutically effective amount of the compositions or vaccines of the invention can include a single treatment or a series of treatments.

As used herein, the term “treatment” is defined as the application or administration of a therapeutic agent described herein, or identified by a method described herein, to a patient, or application or administration of the therapeutic agent to an isolated tissue or cell line from a patient, who has a disease, a symptom of disease or a predisposition toward a disease, with the purpose to cure, heal, alleviate, relieve, alter, remedy, ameliorate, improve or affect the disease, the symptoms of disease, or the predisposition toward disease. Further, the terms “patient” “subject” and “individual” are used interchangeably herein, and mean organism to be treated, such as a mammal, with human patients being preferred. In some cases, the methods of the invention find use in experimental animals, in veterinary applications, and in the development of animal models for disease, including, but not limited to, rodents including mice, rats, and hamsters, as well as non-human primates.

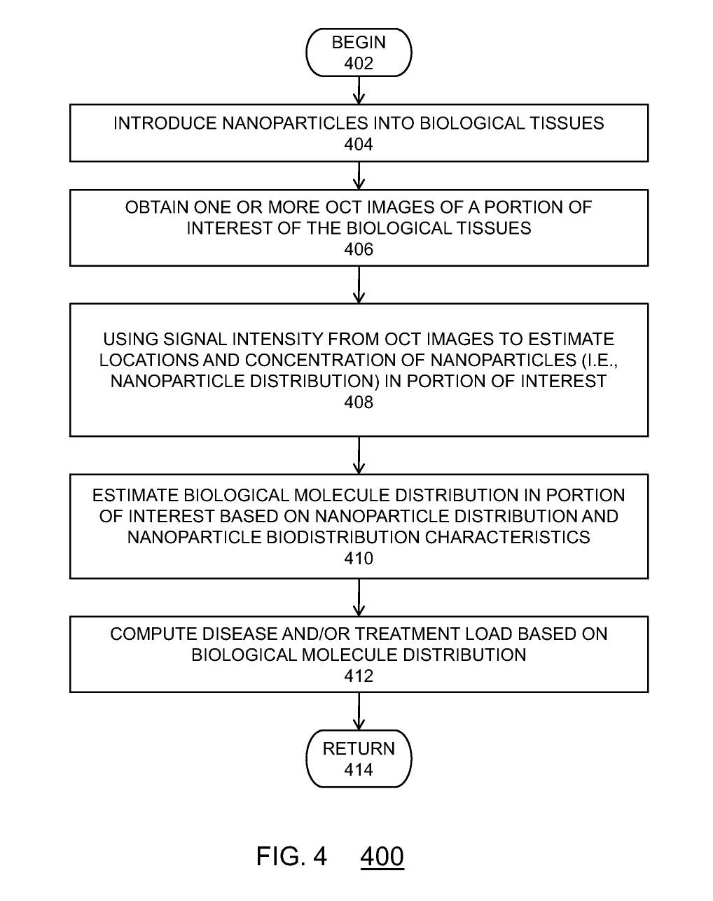

Accordingly, by using nanoparticles conjugated with a targeting molecule, as described above, OCT images can be generated to determine locations and concentrations of biological molecules of interest. This process is described below with respect to FIG. 4.

FIG. 4 is a flowchart of steps in an exemplary method 400 for characterizing biological tissues in accordance with an embodiment of the invention. Method 400 begins at step 402 and continues on to step 404. At step 404, nanoparticles are introduced and/or combined with the biological tissues. As described above, several methods can be used to introduce the nanoparticles in the biological tissues. Further, the size, shape, and composition of the nanoparticles can be selected so as to provide a strong intensity signal at selected operating wavelengths of the OCT system.

After the nanoparticles are combined with the biological tissues at step 404, one or more OCT images of the biological tissues are obtained at step 406. At step 406, at least one OCT image can be obtained using electromagnetic radiation (i.e., light) having a center wavelength corresponding to the strong absorption wavelength for the nanoparticles introduced at step 404. Once the OCT images are obtained at step 406, the signal intensity data from the OCT images can be used at step 408 to estimate the distribution of the nanoparticles in the biological tissues being imaged. In particular, the concentration of nanoparticles in various portions of the biological tissues can be estimated based on the intensity data. For example, as described above, the substantially linear relationship between intensity data and nanoparticle concentration can be used to estimate the nanoparticle distribution at step 408.

Due to the nature of the OCT imaging process, pre-processing of the OCT image data can be required to obtain an accurate estimate of nanoparticle distribution. In particular, as OCT imaging relies on reflection of light penetrating biological tissues, some compensation is needed to accounting for depth. That is, for tissues at the surface of the biological tissues, the reflected light consists mainly of the incident light. In contrast, only a portion of the incident light will penetrate the biological tissues and reflect off structures at lower depths. Accordingly, in some embodiments of the invention, step 408 can also include providing an attenuation and/or normalization of the intensity data to account for the different amounts of reflected light. That is, the intensity data is adjusted to provide substantially similar intensity data for similar types of tissues, regardless of the depth of the tissues. Thereafter, the intensity data associated with the nanoparticles can be extracted to estimate the nanoparticle distribution.

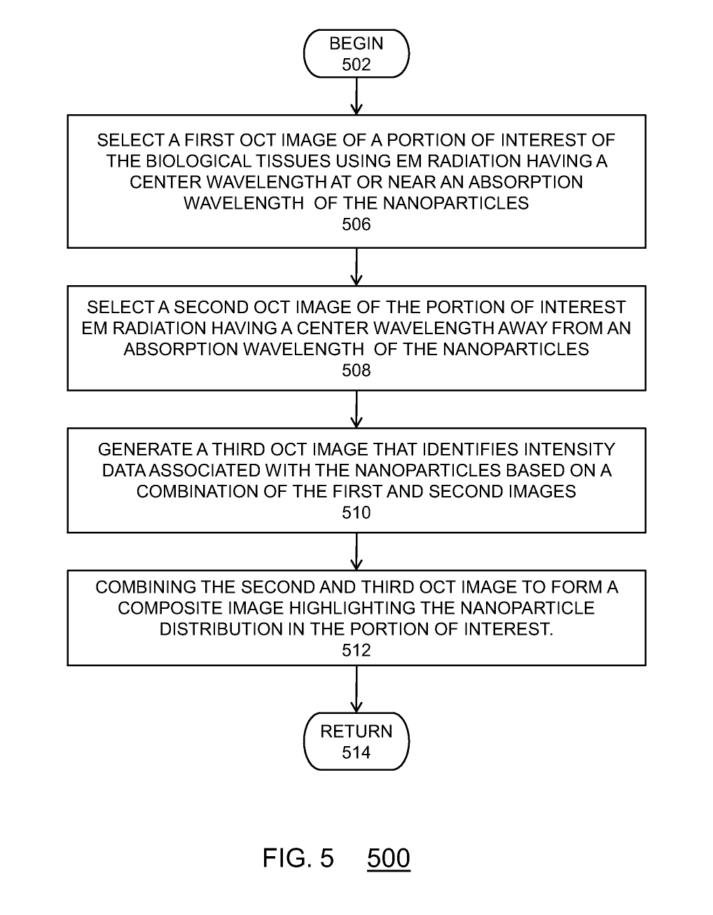

In general, the nanoparticles can be selected to provide OCT intensity data of a magnitude significantly higher than that of the biological structures in the OCT image. Accordingly, in some embodiments, the intensity data associated with the nanoparticles can be selected based on a threshold value. Such a threshold value can be fixed or derived from the OCT image. For example, intensity data of a higher magnitude associated with the nanoparticles can generate a distribution of intensity data points, separate from that associated with the OCT intensity data for the biological tissues. Accordingly, the difference between these distributions can be used to select a threshold value. However, in some instances it can be difficult to differentiate intensity data associated with the nanoparticles from intensity data associated with the biological tissues in the OCT image. That is, the intensity data may not be of a significantly higher magnitude. Accordingly, in some embodiments of the invention, additional processing can be performed to ensure proper identifyication of intensity data associated with the nanoparticles. Such a method is illustrated with respect to FIG. 5.

FIG. 5 is a flowchart of steps in an exemplary method 500 for identifying OCT intensity data associated with nanoparticles in biological tissues. Method 500 begins at step 502 and continues to step 504. At step 504, a first OCT image of the portion of interest of the biological tissues is selected or obtained. This first OCT image is obtained using electromagnetic radiation having a center wavelength at or near the absorption wavelength of the nanoparticles to generate a contrast in the OCT image. Concurrently or separately from step 504, a second OCT image of the portion or interest is also selected or obtained at step 506. In contrast to the first image, the second OCT is obtained at step 506 using electromagnetic radiation having a center wavelength away from the absorption wavelength of the nanoparticles so as not to generate contrast in the OCT image.

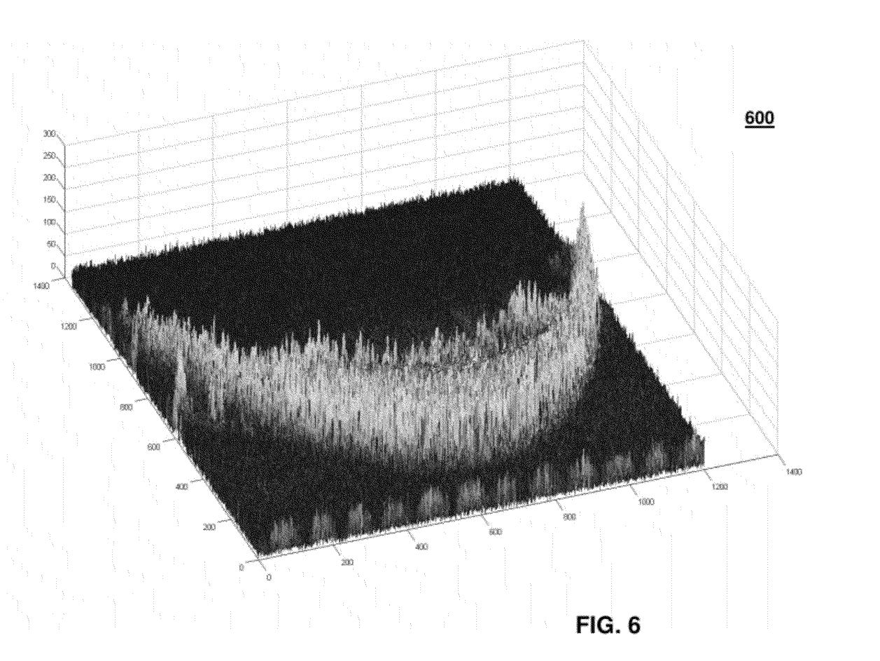

These images obtained at steps 504 and 506 (after any attenuation or normalization) are then used at step 508 to generate a third OCT image. In some embodiments, the third OCT image can be difference between the first and second OCT images. Such subtractions can be performed on a weighted or non-weighted basis. Further, the substractions can be performed for all or a part of the OCT images. Thus, the resulting intensity data set provides effectively increases the signal-to-noise ratio of the intensity data in the first OCT image by using the second OCT image to remove the “noise” caused by the intensity data associated with the biological tissues. As a result, an intensity dataset associated primarily, if not entirely, with the nanoparticles is generated. One such dataset is shown in FIG. 6. FIG. 6 is a three-dimensional (3D) plot 600 of OCT intensity as a function of position for first and second OCT images of a mouse cornea obtained as described above with respect to FIG. 5, where the mouse cornea stroma was injected with 0.1 cc of 50 nM gold nanorods. As shown in FIG. 6, once the intensity data associated with the biological tissues is removed, as described above, the intensity data associated with the nanoparticles is clearly distinguishable.

In some embodiments, the third OCT image can be used to generate an image of solely the intensity data associated with the nanoparticles, as shown in FIG. 6. However, in some embodiments, this identified intensity data can also be used to enhance an OCT image including intensity data associated with biological tissues. For example, the intensity data from the second and third OCT images can be combined at step 510 to obtain an OCT image of the biological tissues highlighting the distribution of the nanoparticles. For example, the intensity data from the second OCT image can be overlaid with the intensity data from the third OCT image. Further, to enhance contrast between the two datasets, a different color palette can be applied to the intensity data from each of the OCT images. As used herein, the term “color palette” refers to the set of colors for an image. In one configuration, the second OCT image can be associated with a grayscale color palette (i.e., colors including black, white, and/or one or more shades of gray therebetween) while the third OCT image can be associated with a non-grayscale color palette (i.e., colors including one or more shades of any other colors, separately or in combination with grayscale, black, or white colors). Accordingly, the structures of the biological tissues are presented for reference while the distribution of the nanoparticles in the biological tissues is highlighted. Method 500 can then resume previous processing at step 512, including repeating method 500 or returning to method 400.

Referring back to FIG. 4, once the nanoparticle intensity data is used to determine the nanoparticle distribution at step 408, the nanoparticle distribution can be used to estimate a biological molecule distribution in the biological tissues at step 410. Although the nanoparticle concentration can be directly obtained from the intensity data, this nanoparticle distribution may not directly correlate to the concentration of biological molecules in the biological tissues. For example, if the nanoparticles are injected into the bloodstream of a patient, the result intensity signal at a specific point in the patient will generally be based on two primary factors. First, the number of biological molecules at the specific point defines the number of available locations for nanoparticles to bind to. Second, the distribution of the nanoparticles in the patient resulting from flow of the bloodstream (i.e., the biodistribution of the nanoparticles) defines the number of nanoparticles the specific point is exposed to. Accordingly, even though a portion of biological tissues may have a large number of biological molecules of interest, the biodistribution of the nanoparticles may result in these biological molecules being exposed to a small number of nanoparticles. As a result, only a small number of nanoparticles may bind, resulting in a relatively low OCT intensity signal. Similarly, even though a portion of biological tissues may have a small number of biological molecules of interest, the biodistribution of the nanoparticles may result in these biological molecules being exposed to a large number of nanoparticles. As a result, only a large number of nanoparticles may bind, resulting in a relatively high OCT intensity signal.

To account for such variation in the bio distribution of the nanoparticles, the biodistribution is used at step 410 to estimate the concentration of biological molecules. For example, if a normalized OCT signal intensity at two different points is ˜1, and the biodistribution or uptake factor in each of these regions is 0.3 and 0.5, then the relative count of biological molecules in these two regions can be estimated as 1÷0.3=3.3 and 1÷0.5=2, respectively. Accordingly, even though the intensity data may be the same for each region, the estimated number of biological molecules will be different: more in the first region than in the second region. As used herein, the term “uptake” refers to the average number of nanoparticles present in a region of a biological tissue.

The bio-distribution of nanoparticles in a living subject can be evaluated in multiple ways, but typically requires a baseline scan that estimates the distribution of the particles in various tissues of the body. For example, by administering the typical nanoparticle dose to a healthy subject, a baseline estimation for the distribution of the particle is achieved from the basic OCT images. Specifically, to give an estimation for the ability of the nanoparticle to reach various tissues. Therefore, if in a particular tissue in a diseased patient, more OCT signal is recorded, the operator can infer that the nanoparticles are selectively binding to that area, can from the baseline biodistribution images, estimate the concentration of target proteins in that tissue.

In the example above, it is assumed that the likelihood that a nanoparticle exposed to an unbound biological molecule will be 100% will bind or attached thereto (i.e., the nanoparticle has a sticking coefficient of 1 with respect to the biological molecule). However, in some instances, this likelihood may be less than 100%. Further, this likelihood can vary locally. Accordingly, global and/or local variations in the sticking coefficient of the nanoparticles can be used to further adjust the relative counts of the biological molecules. For example, if the two regions discussed above have overall (global plus local effects) sticking coefficients of 0.4 and 0.7, the relative counts of biological molecules in the two regions would then be estimated as 1÷0.3×0.4=1.3 and 1÷0.5×0.7=1.4, respectively. Similarly, other factors can affect resulting nanoparticle concentrations and thus the estimates of biological molecule concentration.

Once the distribution of the biological molecules is obtained at step 410, a disease load can be estimated at step 412. That is, the estimate of the distribution of biological molecules can be used to characterize the state of the biological tissues. Such a characterization can be performed at step 412 in several ways. For example, the number of biological molecules can be used to categorize the disease state. In such a configuration, one or more threshold values can be specified that associate state of a disease with a number of biological molecules. In some instances, this categorization can be further based on localized data. That is, a disease state may be associated with a number of biological molecules in a particular location in the biological tissues. For example, a large number of biological molecules in the cornea may indicate macular degeneration. Thereafter, the method 400 can proceed to step 414 and resume previous processing, including repeating method 400.

In some embodiments the nanoparticles can be used to deliver therapies, as described above. That is, the nanoparticles can be coated with drugs or other treatment compounds for directly treating the biological tissues of interest. Accordingly, as the tissues are treated, nanoparticles will detach over time (due to bonds breaking or removal of molecular target by the treatment compound). Consequently, the treatment load can also be monitored using the methods described in method 400. In particular, a first OCT image can be obtained to estimate an initial treatment load (i.e., amount of treatment compound×concentration of nanoparticles). Thereafter, additional OCT images can be used to estimate the remaining number/concentration of nanoparticles and thus the subsequent treatment efficacy and/or disease load.

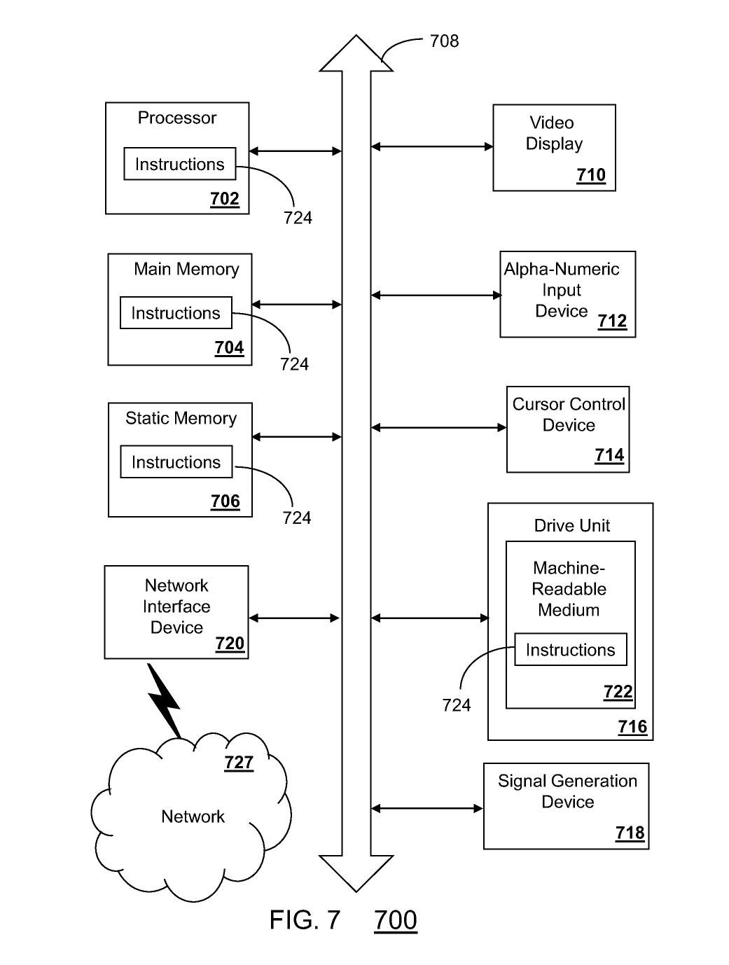

FIG. 7 is a schematic diagram of a computer system 700 for executing a set of instructions that, when executed, can cause the computer system to perform one or more of the methodologies and procedures described above. In some embodiments of the present invention, the computer system 700 operates as a standalone device. In other embodiments of the present invention, the computer system 700 can be connected (e.g., using a network) to other computing devices. In a networked deployment, the computer system 700 can operate in the capacity of a server or a client developer machine in server-client developer network environment, or as a peer machine in a peer-to-peer (or distributed) network environment.

The machine can comprise various types of computing systems and devices, including a server computer, a client user computer, a personal computer (PC), a tablet PC, a laptop computer, a desktop computer, a control system, a network router, switch or bridge, or any other device capable of executing a set of instructions (sequential or otherwise) that specifies actions to be taken by that device. It is to be understood that a device of the present disclosure also includes any electronic device that provides voice, video or data communication. Further, while a single computer is illustrated, the phrase “computer system” shall be understood to include any collection of computing devices that individually or jointly execute a set (or multiple sets) of instructions to perform any one or more of the methodologies discussed herein.

The computer system 700 can include a processor 702 (such as a central processing unit (CPU), a graphics processing unit (GPU, or both), a main memory 704 and a static memory 706, which communicate with each other via a bus 708. The computer system 700 can further include a display unit 710, such as a video display (e.g., a liquid crystal display or LCD), a flat panel, a solid state display, or a cathode ray tube (CRT)). The computer system 700 can include an alphanumeric input device 712 (e.g., a keyboard), a cursor control device 714 (e.g., a mouse), a disk drive unit 716, a signal generation device 718 (e.g., a speaker or remote control) and a network interface device 720.

The disk drive unit 716 can include a computer-readable medium 722 on which is stored one or more sets of instructions 724 (e.g., software code) configured to implement one or more of the methodologies, procedures, or functions described herein. The instructions 724 can also reside, completely or at least partially, within the main memory 704, the static memory 706, and/or within the processor 702 during execution thereof by the computer system 700. The main memory 704 and the processor 702 also can constitute machine-readable media.

Dedicated hardware implementations including, but not limited to, application-specific integrated circuits, programmable logic arrays, and other hardware devices can likewise be constructed to implement the methods described herein. Applications that can include the apparatus and systems of various embodiments of the present invention broadly include a variety of electronic and computer systems. Some embodiments of the present invention implement functions in two or more specific interconnected hardware modules or devices with related control and data signals communicated between and through the modules, or as portions of an application-specific integrated circuit. Thus, the exemplary system is applicable to software, firmware, and hardware implementations.

In accordance with various embodiments of the present invention of the present disclosure, the methods described herein can be stored as software programs in a computer-readable medium and can be configured for running on a computer processor. Furthermore, software implementations can include, but are not limited to, distributed processing, component/object distributed processing, parallel processing, virtual machine processing, which can also be constructed to implement the methods described herein.

The present disclosure contemplates a computer-readable medium containing instructions 724 or that receives and executes instructions 724 from a propagated signal so that a device connected to a network environment 726 can send or receive voice and/or video data, and that can communicate over the network 726 using the instructions 724. The instructions 724 can further be transmitted or received over a network 726 via the network interface device 720.

While the computer-readable medium 722 is shown in an exemplary embodiment to be a single storage medium, the term “computer-readable medium” should generally be taken to include a single medium or multiple media (e.g., a centralized or distributed database, and/or associated caches and servers) that store the one or more sets of instructions. The term “computer-readable medium” shall also be taken to include any tangible medium or device that is capable of storing, encoding or carrying a set of instructions for execution by the machine and that cause the machine to perform any one or more of the methodologies of the present disclosure.

The term “computer-readable medium” shall accordingly be taken to include, but not be limited to, solid-state memories such as a memory card or other package that houses one or more read-only (non-volatile) memories, random access memories, or other re-writable (volatile) memories; magneto-optical or optical medium such as a disk or tape; as well as devices including a tangible transmission medium for carrying carrier wave signals such as a signal embodying computer instructions; and/or a digital file attachment to e-mail or other self-contained information archive or set of archives considered to be a distribution medium equivalent to a tangible storage medium. Accordingly, the disclosure is considered to include any one or more of a computer-readable medium or a distribution medium, as listed herein and to include recognized equivalents and successor media, in which the software implementations herein are stored.

Although the present specification describes components and functions implemented in the embodiments of the present invention with reference to particular standards and protocols, the disclosure is not limited to such standards and protocols. Each of the standards for Internet and other packet switched network transmission (e.g., TCP/IP, UDP/IP, HTML, and HTTP) represent examples of the state of the art. Such standards are periodically superseded by faster or more efficient equivalents having essentially the same functions. Accordingly, replacement standards and protocols having the same functions are considered equivalents.

Applicants present certain theoretical aspects above that are believed to be accurate that appear to explain observations made regarding embodiments of the invention. However, embodiments of the invention may be practiced without the theoretical aspects presented. Moreover, the theoretical aspects are presented with the understanding that Applicants do not seek to be bound by the theory presented.

While various embodiments of the invention have been described above, it should be understood that they have been presented by way of example only, and not limitation. Numerous changes to the disclosed embodiments can be made in accordance with the disclosure herein without departing from the spirit or scope of the invention. Thus, the breadth and scope of the invention should not be limited by any of the above described embodiments. Rather, the scope of the invention should be defined in accordance with the following claims and their equivalents.

Although the invention has been illustrated and described with respect to one or more implementations, equivalent alterations and modifications will occur to others skilled in the art upon the reading and understanding of this specification and the annexed drawings. In addition, while a particular feature of the invention may have been disclosed with respect to only one of several implementations, such feature may be combined with one or more other features of the other implementations as may be desired and advantageous for any given or particular application.

The terminology used herein is for the purpose of describing particular embodiments only and is not intended to be limiting of the invention. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

Unless otherwise defined, all terms (including technical and scientific terms) used herein have the same meaning as commonly understood by one of ordinary skill in the art to which this invention belongs. It is further understood that terms, such as those defined in commonly used dictionaries, should be interpreted as having a meaning that is consistent with their meaning in the context of the relevant art and will not be interpreted in an idealized or overly formal sense unless expressly so defined herein.

What is claimed is:

1. A method of characterizing biological tissues, the method comprising:
obtaining a first optical coherence tomography (OCT) image of a selected portion of biological tissues combined with a plurality of nanoparticles, the plurality of nanoparticles configured to bind to one or more types of biological molecules in the biological tissues and to produce contrast during OCT imaging;
estimating a first distribution of the plurality of nanoparticles in the selected portion based on the first OCT image; and
characterizing the selected portion with respect to the types of biological molecules based on the distribution.

2. The method of claim 1, wherein the step of estimating further comprises:
identifying intensity data in the first OCT image associated with the plurality of nanoparticles;
computing as the first distribution a quantity and a location of the plurality of nanoparticles in the selected portion based on the identified intensity data and biodistribution characteristics of the plurality of nanoparticles with respect to the biological tissues.

3. The method of claim 1, wherein the step of characterizing further comprises:
computing a disease load associated with the types of biological molecules based on the distribution in the selected portion.

4. The method of claim 1, wherein the step of characterizing further comprises:
computing a treatment load associated with the types of biological molecules based on the distribution in the selected portion.

5. The method of claim 1, wherein the method further comprises:
prior to the step of characterizing, obtaining a second OCT image of the selected portion and estimating a second distribution of the plurality of nanoparticles in the selected portion based on the second OCT image, and
wherein the characterizing further comprises computing a disease load based on a comparison of the first and second distributions.

6. The method of claim 1, wherein the method further comprises:
prior to the step of characterizing, combining an additional plurality of nanoparticles with the biological tissues, obtaining a second OCT image of the selected portion, and estimating a second distribution of the additional plurality of nanoparticles in the selected portion based on the second OCT image, and
wherein the characterizing further comprises computing a disease load based on a comparison of the first and second distributions.

7. The method of claim 1, further comprising introducing the plurality of nanoparticles into the biological tissues.

8. The method of claim 7, further comprising selecting a composition of the plurality of nanoparticles to comprise at least one of metal, a semiconductor, or small molecules.

9. The method of claim 7, further comprising selecting a composition of the plurality of nanoparticles to comprise at least one metal selected from the group consisting of gold, silver, iron oxide, nickel, and cobalt.

10. The method of claim 7, further comprising selecting the plurality of nanoparticles to comprise a plurality of gold nanorods.

11. The method of claim 10, wherein the plurality of gold nanorods are selected to have a length between 20 nm and 60 nm, a diameter between 5 nm and 20 nm.

12. The method of claim 10, further comprising selecting the plurality of gold nanorods to have a optical extinction coefficient between 6×10⁸ M⁻¹cm⁻¹ and 7×10⁸ M⁻¹cm⁻¹ for wavelengths between 500 and 1500 nm.

13. The method of claim 7, further comprising selecting the plurality of nanoparticles comprise at least one targeting component selected from group consisting of an antibody, a minibody, a diabody, an aptamer, an affibody, a peptide, and siRNA.

14. The method of claim 7, wherein the types of biological molecules comprise at least one of a protein, a receptor, an enzyme, a cell, a gene, a bacteria, and a virus.

15. The method of claim 7, wherein the types of biological molecules comprise at least one of an anti-angiogenic factor, a neural factor, a tumor antigen and an anti-inflammatory factor.

16. The method of claim 1, wherein the step of obtaining further comprises exposing at least the selected portion to electromagnetic radiation having a center wavelength between ultraviolet and near-infrared wavelengths.

17. The method of claim 1, wherein the biological tissues comprise ophthalmic tissues.

18. A method of characterizing biological tissues combined with a plurality of nanoparticles configured to bind to one or more types of biological molecules in the biological tissues, the method comprising:
obtaining a first intensity dataset for a first optical coherence tomography (OCT) image of a selected portion of the biological tissues using electromagnetic radiation with a center wavelength substantially equal to a peak absorption wavelength of a plurality of nanoparticles;
obtaining a second intensity dataset for a second OCT image of the selected portion using electromagnetic radiation with a center wavelength substantially unequal to a peak absorption wavelength of a plurality of nanoparticles;
generating a third intensity dataset defining a third OCT image of the selected portion based on a combination of the first and second intensity datasets, the third intensity dataset indicating the areas of the selected portion comprising one or more of the plurality of nanoparticles bound to the types of biological molecules.

19. The method of claim 18, wherein the step of generating further comprises computing a difference between the first and second intensity datasets to define the third intensity dataset.

20. The method of claim 19, where the step of generating further comprises adjusting at least one of the first and second intensity datasets prior to computing the difference.

21. The method of claim 19, wherein the step of generating further comprises attenuating at least one of the first and second intensity datasets prior to computing the difference.

22. The method of claim 18, further comprising:
associating color information with the second and third intensity datasets; and
generating a composite OCT image of the biological tissues based on the color information associated with the second and third intensity datasets,
wherein a color palette for the color information associated with the second intensity dataset and a color palette for the color information associated with the third intensity dataset are selected to be different.

23. The method of claim 22, further comprising selecting a grayscale color palette for the second intensity dataset and selecting a non-grayscale color palette for the third intensity dataset.

24. The method of claim 22, wherein the step of generating a composite OCT image further comprises overlaying the color information for the third intensity dataset over the color information for the second intensity dataset.

25. A system for characterizing biological tissues combined with a plurality of nanoparticles configured to bind to one or more types of biological molecules in the biological tissues and to produce contrast during optical coherence tomography (OCT), the system comprising:
a storage element for storing data associated with OCT images of a selected portion of the biological tissues; and
a processing element communicatively coupled to the storage element, the processing element configured for:
estimating a first distribution of the plurality of nanoparticles in the selected portion based on the data associated with a first OCT image; and
characterizing the selected portion with respect to the types of biological molecules based on the first distribution.

26. The system of claim 25, wherein processing element is further configured during the estimating for:
identifying intensity data in the first OCT image associated with the plurality of nanoparticles;
computing as the first distribution a quantity and a location of the plurality of nanoparticles in the selected portion based on the identified intensity data and biodistribution characteristics of the plurality of nanoparticles with respect to the biological tissues.

27. The system of claim 25, wherein processing element is further configured during the characterizing for:
computing a disease load associated with the types of biological molecules based on the distribution in the selected portion.

28. The system of claim 25, wherein processing element is further configured during the characterizing for:
computing a treatment load associated with the types of biological molecules based on the distribution in the selected portion.

29. The system of claim 25, wherein the processing element is further configured for obtaining a second OCT image of the selected portion and estimating a second distribution of the plurality of nanoparticles in the selected portion based on the second OCT image prior to the characterizing of the selected portion, and wherein the processing element is further configured during the characterizing for computing a disease load based on a comparison of the first and second distributions.

30. A system for characterizing biological tissues combined with a plurality of nanoparticles configured to bind to one or more types of biological molecules in the biological tissues and to produce contrast during optical coherence tomography (OCT), the system comprising:
a storage element for storing a first intensity dataset for a first OCT image of a selected portion of the biological tissues obtained using electromagnetic radiation with a center wavelength substantially equal to a peak absorption wavelength of a plurality of nanoparticles and a second intensity dataset for a second OCT image of the selected portion obtained using electromagnetic radiation with a center wavelength substantially unequal to a peak absorption wavelength of a plurality of nanoparticles; and
a processing element communicatively coupled to the storage element, the processing element configured for:
generating a third intensity dataset defining a third OCT image of the selected portion based on a combination of the first and second intensity datasets, the third intensity dataset indicating the areas of the selected portion comprising one or more of the plurality of nanoparticles bound to the types of biological molecules.

31. The system of claim 30, wherein processing element is further configured during the generating for computing a difference between the first and second intensity datasets to define the third intensity dataset.

32. The system of claim 31, wherein the processing element is further configured during the generating for adjusting at least one of the first and second intensity datasets prior to computing the difference.

33. The system of claim 31, wherein the processing element is further configured during the generating for attenuating at least one of the first and second intensity datasets prior to computing the difference.

34. The system of claim 30, wherein the processing element is further configured for:
associating color information with the second and third intensity datasets; and
generating a composite OCT image of the biological tissues based on the color information associated with the second and third intensity datasets,
wherein a color palette for the color information associated with the second intensity dataset and a color palette for the color information associated with the third intensity dataset are selected to be different.

35. The system of claim 34, wherein the processing element is further configured for associating a grayscale color palette with the second intensity dataset and a non-grayscale color palette for the third intensity dataset.

36. The method of claim 30, wherein the processing element is further configured for generating the composite OCT image by overlaying the color information for the third intensity dataset over the color information for the second intensity dataset.

37. The method of claim 7, wherein the types of biological molecules comprise at least one of VEGFR1, VEGFR2, αvβ3, CCR3, soluble VEGF, PEDF, and PDGF.
